(19) United States
(12) Patent Application Publication
(10) Pub. No.: US 2014/0163678 A1
(43) Pub. Date: Jun. 12, 2014

(54) METHODS FOR AUGMENTING OR RECONSTRUCTING A BREAST AND A KIT THEREFOR

(71) Applicant: ALLERGAN, INC., Irvine, CA (US)

(72) Inventor: Dennis Van Epps, Goleta, CA (US)

(73) Assignee: ALLERGAN, INC., Irvine, CA (US)

(21) Appl. No.: 13/711,978

(22) Filed: Dec. 12, 2012

(51) Int. Cl. A61F 2/12

(52) U.S. Cl. CPC A61F 2/12
USPC 623/8

(57) ABSTRACT

Methods and systems are provided for facilitating a breast augmentation or reconstruction procedure. The methods generally include performing an incision in a breast of a patient and forming a surgical pocket in the breast, introducing, into the pocket, a mock breast implant, introducing an injectable composition adjacent the mock breast implant in the breast, and replacing the mock implant with a permanent breast implant. Kits useful to perform these methods are also provided.

The present invention generally relates to plastic surgery procedures and more specifically relates to methods for augmenting or reconstructing a breast and a kit therefor.

### BACKGROUND

Soft tissue replacement methods are commonly used for a wide variety of clinical and cosmetic purposes. One use involves reconstructive applications that rebuild and restore a body part or structure to correct deformities from congenital anomalies, trauma, cancer, infections, disease, or medication side effects. The replacement tissue serves to support surrounding tissue and to maintain the normal appearance of the body. The restoration of this normal appearance has a significant beneficial psychological effect on post-operative patients, alleviating much of the shock and depression that often follows extensive surgical procedures. Another use involves augmentation applications that alter a body part or structure usually to improve its cosmetic or aesthetic appearance. Augmentation of the appearance also has beneficial psychological effects that improve self-esteem, well-being, and confidence of an individual.

The use of implantable prosthesis for breast shaping, for example, for breast reconstruction following traumatic or surgical loss of breast tissue or, electively to increase volume of the breast is well known. Typically, the prosthesis or implant comprises of a soft, flexible envelope containing a liquid or gelatinous material. The envelope is commonly made from silicone or other bio-compatible polymer with varying degrees of elastic memory and permeability. These prostheses are filled with saline, and silicone oil or gel to mimic the tone and feel of natural breast tissue.

Fat grafting, or lipoinjection, in breast augmentation and reconstruction procedures are also well known. In these procedures, autologous adipose tissue, typically obtained through liposuction procedures which remove fat from a donor site such as a thigh or abdomen of a patient, is aseptically collected, and sometimes processed and purified, and, thereafter, reintroduced into the patient by injection into the breast area. The fat may be introduced into multiple areas of the breast, for example, the upper and lower outer quadrant. However, these procedures are not ideal for significant long term breast volumizing.

Injectable fillers, such as hyaluronic acid-based compositions developed for restoring facial skin volume and reducing wrinkles, have also been studied for implantation in the breast.

The present invention provides a method and kit for use in breast augmentation and/or reconstruction using both a breast prosthesis and an injectable such as, but limited to, autologous fat.

### SUMMARY

Accordingly, a system and method of performing breast augmentation or reconstruction is provided. The present invention addresses the need for better systems and methods for use in breast surgery procedures.

It has become increasingly more popular to augment the appearance of the breast using autologous fat or other biocompatible injectable substances, such as stabilized hyaluronic acid based hydrogels. By injecting such substances into the breast, minor imperfections, divots, asymmetries and other conditions affecting breast appearance can be corrected without major surgery.

More recently, the use of injectable substances has sometimes been combined with traditional breast implant surgery, that is, the well known procedure in which a silicone gel-filled or saline filled breast prosthesis is introduced into a breast to provide volume, replace lost tissue or improved firmness of the breast. When these procedures are used in combination, the surgeon may first implant a breast prosthesis in a conventional manner, and then later add additional shape, volume or correction to the implanted breast by introducing an injectable composition into the implanted breast, for example, in regions that would benefit from increased fullness or contouring.

In this way, by using both of these well know procedures in combination, a surgeon can provide the desired volume, through the implant, and additional detailed contouring or correction using the injectable, to create a more customized aesthetic outcome, closely in line with the patient's wishes.

However, it can be appreciated that during such procedure as generally described, the step of introducing the injectable, whether it be the patient's own adipose tissue or an commercial injectable, can potentially cause damage to the implant. Even when a blunt-tipped cannula is utilized for the injection step, any instrument introduced into the implanted breast can potentially tear, pierce or rupture the implant. Obviously, any compromised integrity to the implant most preferably should be avoided.

The method of the present invention provides a method for augmenting or reconstructing a breast, using a combination of an implant and an injectable, but with far less potential for damaging the implant.

The method generally comprises the steps of performing an incision in a breast of a patient and forming a surgical pocket; introducing, through the incision, a mock breast implant into the surgical pocket; introducing an injectable filler composition into the implanted breast, for example, using a blunt ended cannula; and replacing the mock implant with a permanent breast implant. In accordance with one embodiment, after the mock implant is introduced, the incision may be temporarily closed, for example, with sutures, and the injectable is introduced to more precisely contour or provide further volume to the implanted breast. After introduction of the injectable, the temporary surgical incision is reopened, the mock implant is removed from the surgical pocket and the permanent implant is carefully implanted in the cavity left from the removed mock implant. Prior to introduction of the permanent implant, the surgical pocket may be inspected and cleared of any injectable that may have penetrated the pocket. The surgical incision is then sealed, leaving the filler composition and permanent breast implant in place in the breast. The breast implant may comprise any conventional breast implant suitable for this procedure. The injectable composition may comprise adipose tissue, a hyaluronic acid- or collagen-based commercial soft tissue filler, other suitable biocompatible injectable material, active agents or vitamins, or even a combination of fat and commercial soft tissue filler.

In another aspect of the invention, a system, for example, in the form of a surgical kit, is provided to facilitate a combination implant and injectable breast augmentation or reconstruction procedure. The kit may comprise a mock breast implant; a permanent breast implant; an assembly or device for introducing an injectable filler into the breast; and instructions for use, for example, in electronic or written form. The instructions may include instruction to use the mock implant as a space-holder in a breast during a fat grafting or other injection-based procedure, and instruction to replace the mock implant with the permanent breast prosthesis after the injection procedure. The mock implant may comprise a fluid-fillable, for example, saline-fillable, elastomeric envelope or shell having an appropriately matched size relative to the permanent implant.

Each and every feature described herein, and each and every combination of two or more of such features, is included within the scope of the present invention provided that the features included in such a combination are not mutually inconsistent.

### BRIEF DESCRIPTION OF THE DRAWINGS

These and other aspects, features and advantages of the present invention may be more clearly understood with reference to the following Detailed Description when considered in conjunction with the accompanying Drawings, which show:

FIG. 1 is simplified diagram of a system or kit in accordance with an embodiment of the invention; and

FIG. 2 is simplified diagram of a system or kit in accordance with another embodiment of the invention.

### DETAILED DESCRIPTION

In one aspect of the invention, a method of facilitating a breast augmentation or reconstruction procedure is provided.

The method generally comprises the steps of performing an incision in a breast of a patient and forming a surgical pocket for receiving a breast implant, and introducing, through the incision and into the pocket, a mock breast implant.

The mock implant may be of any suitable structure and materials, but generally is constructed to be suitable for temporary implantation in a breast. In some embodiments, the mock implant closely matches the shape and size of a conventional permanent breast implant, for example, a permanent silicone or saline-filled implant to be introduced later in the procedure. The mock implant may comprise a soft solid material, for example, a polyurethane foam, or a solid silicone gel. Alternatively, the mock implant may comprise a flexible envelope or shell and a flowable material, for example, saline or a hydrogel, enclosed therein. In some embodiments, the mock implant comprises an unfilled, hollow envelope or shell that may be filled just before or after implantation in the breast. In some embodiments, the mock implant is a hollow structure that is not intended to be filled with liquid, but which will substantially retain its shape and will support a patient's tissue without substantially collapsing after implantation in the breast.

In certain embodiments of the invention in which the mock implant comprises a shell or envelope filled with a liquid or gel, the mock implant may comprise a puncture resistant, puncture proof, and/or tear resistant envelope or shell, in order to resist or prevent damage thereto by a cannula, for example a blunt-tipped cannula, used to introduce fat or injectables adjacent the mock implant.

For example, the shell may comprise a flexible silicone wall and reinforcement means, such as, but not limited to, a nylon other suitable polymer mesh embedded within the silicone wall.

The mock implant may be substantially identical in shape and structure to a conventional permanent, gel or saline filled implant, however with the additional feature of being reinforced so that it is tear and/or puncture proof or tear and/or puncture resistant.

After implanting of the mock implant, the incision may be sealed, for example, by suturing or surgical tape or adhesive. The sealing of the incision may be sufficiently secure so as to hold the mock implant securely in place in the breast during lifting of the patient into a seated position. After sealing the incision, the physician may then observe the mock implant-implanted breast to determine where further volume or shaping of the breast is desirable in view of the patient's desired aesthetic goals delineated prior to the surgery. This step may include having the patient raised from the supine position into a more upright position, to facilitate visualization of the implanted breast when the patient is in natural standing position. As will be appreciated by the surgeon, intraoperative tissue swelling resulting from the implant procedure will be taken into consideration during this step, as reduction in swelling after surgery may alter the final appearance of the breast.

Next, the physician introduces an injectable filler composition to provide such further volume or contouring, for example, for correction of divots or recessed regions, to cover and conceal any potential seams of the final breast implant that may be visible in a thin patient, for example, or to provide more volume in specific regions, for example, in the lower or upper outer quadrants of the breast, or to further enhance the tone or appearance of the implanted breast. In one embodiment, the injectable is introduced in the breast tissue surrounding or near the mock implant without penetrating into the surgical pocket. Naturally, the amount of injectable filler introduced during this step will depend on the desired aesthetic outcome, but any suitable amount of the injectable may be introduced, for example, about 1.0 cc up to 10 cc or greater, and through one or more injection pathways. The injection step may be performed with a conventional cannula, for example, a blunt tipped cannula, for example.

Once the implanted breast exhibits the desired volume and shape, the original incision may be carefully reopened, and the mock implant is explanted from the breast. Following the removal of the mock implant, the pocket may be inspected or checked and cleaned, for example, by removing any fat, or other injectable, that has penetrated into the pocket. The mock implant can then be placed aside to be sterilized for additional use, or may be disposed of. At this time, a permanent breast implant is introduced into the pocket, with the injectable remaining in place in the area surrounding the pocket. The surgeon then closes the incision and completes the surgery in a conventional manner.

The injectable composition introduced into the breast along with the mock implant may be any suitable, biocompatible, injectable.

In some embodiments of the invention, the injectable is fat, or lipoaspirate from the patient's own body. In this instance, the method of the invention may therefor also include the step of removing fat tissue from a donor region of the patient, for example, from the thighs, buttocks or abdomen. The removed fat, or lipoaspirate, can then be introduced into the breast with or without pre-processing of the fat. In some embodiments, the fat is removed from the donor site immediately prior to the making of the surgical breast pocket. Removal of the fat is performed using standard liposuction procedures modified if needed to maintain or enhance viability of the fat tissue. In some instances, the fat is mixed with a composition designed to enhance viability or health of the fat in order to improve longevity success of the fat grafting procedure. The composition that can be mixed with the fat may be, for example, a hyaluronic acid and collagen-based composition, such as a composition described in co-owned U.S. patent application Ser. No. 13/605,565, filed on Sep. 6, 2012, U.S. patent application Ser. No. 13/603,213, filed on Sep. 4, 2012 and U.S. Provisional Patent Application No. 61/586,589, filed on Jan. 13, 2012, the entire contents of each of these documents being incorporated herein by this specific reference. Alternatively, the composition may be a composition comprising active agents for enhancing viability of fat mixed therewith.

Alternative to the injectable comprising lipoaspirate or fat tissue, the injectable may be any other suitable injectable composition that can be used to sculpt, shape, volumize, augment, correct, repair or improve the health of a breast in the augmentation or reconstruction procedure.

It should be appreciated that the term “permanent implant” includes any breast implant that is primarily intended to remain in the breast for a significant length of time, for example, for the life of the patient, including breast implants that are commonly expected to be removed and replaced, as is sometimes recommended, every ten to fifteen years, and those that are replaced for elective reasons such as a desire to change to a smaller or larger size.

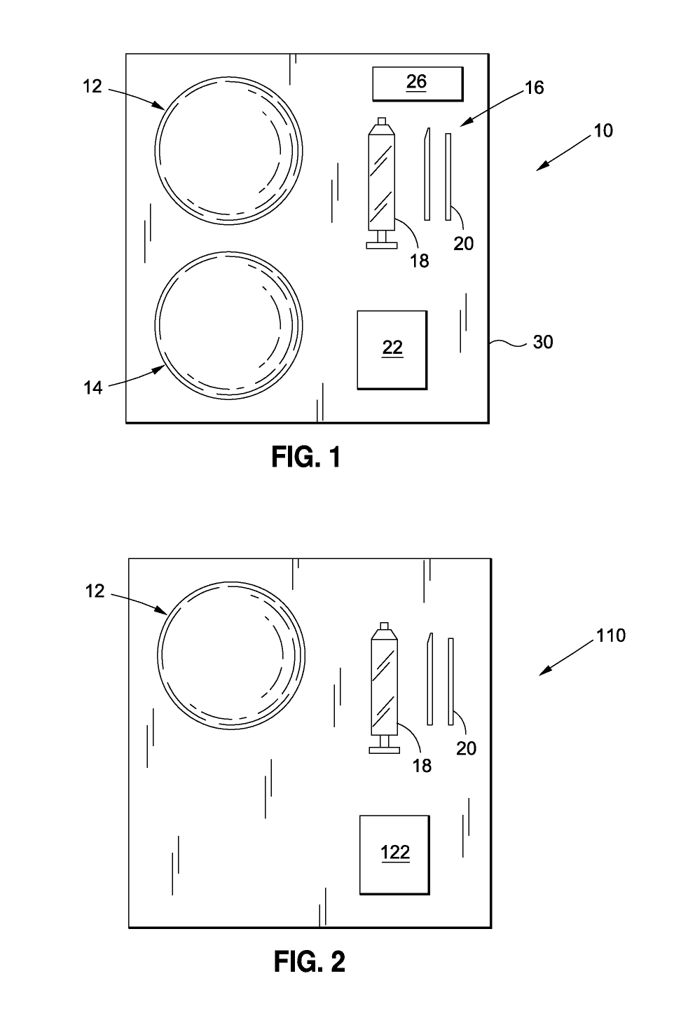

Turning now to FIG. 1, a kit 10 is shown in accordance with another aspect of the invention. The kit 10 is useful for facilitating a breast augmentation or reconstruction procedure involving a conventional breast implant (e.g. saline or gel filled soft tissue implant) in combination with an injectable composition (e.g. injectable soft tissue fillers including adipose tissue, hydrogels including known commercial dermal fillers and other soft tissue sculpting compositions, compositions comprising primarily vitamins and/or active agents, and various combinations thereof).

In the shown embodiment, the kit 10 generally comprises a mock breast implant 12 as described elsewhere herein, a permanent breast implant 14, a injection assembly 16, for example, one or more instruments, for example, a syringe 18 and one or more cannulas, for example, a blunt tipped cannula 20, for introducing an injectable into the breast, and instructions 22 for using the mock implant 12 as a space-holder in a breast during an augmentation or reconstruction procedure.

In a specific embodiment, the injection assembly 16 is a fat grafting assembly 16, that is, instruments 18, 20 may be useful for performing a fat grafting procedure.

Optionally, the fat grafting assembly 16 may further include an injectable composition 26, for example, a composition that can be combined or mixed with fat from a donor site, such as the hips or thighs of the patient prior to being introduced into the breast, as described elsewhere herein.

The mock implant 12, permanent implant 14 and injection assembly 16 may all be hermetically sealed and contained in a container 30. Each kit 10 may be designed for use in reconstructing or augmenting one breast of a patient. In an alternative embodiment, not shown, each kit is designed for use in reconstructing or augmenting both breasts of a patient. It can then be expected that, for bilateral breast surgery, two mock implants, and two permanent implants may be included in the kit.

In some embodiments of the invention, the mock implant 12 is shaped and sized to match the permanent breast implant 14. In some instances, the mock implant 12 and the permanent breast implant 14 are structurally different, for example, in terms of materials of construction, while being similar or identical in size and shape. The mock implant 12, as described above, is structured to be a temporary space-holder in the breast as mentioned herein, and thus may have a very different structure than a permanent breast implant 14.

For example, in some embodiments, the mock implant 12 comprises one of a polyurethane foam member, a saline-filled elastomeric envelope, or a soft solid gel member, having a size and shape substantially similar to the permanent breast implant 14. In other embodiments, the mock implant 12 is an empty shell of appropriate size and shape which can be filled after positioning in the breast. The permanent implant 14 may be, for example, a silicone gel filled, conventional round or shaped breast implant, such as a breast implant marketed under the trademark Natrelle® and available from Allergan, Inc. The permanent implant 14 can be any suitable permanent implant, for example, commercially available permanent breast implant, for example, an empty, saline fillable shell, or a saline-filled or gel-filled breast implant.

Turning now to FIG. 2, in yet another embodiment, a kit 110 is provided which is substantially identical to kit 10, with one major difference being that kit 110 does not contain a permanent breast implant. Kit 110 generally comprises a mock breast implant 12, a injection assembly 16, for example, one or more fat grafting instruments, for example, a syringe 18 and a blunt tipped cannula 20, and instructions 122 for using the mock implant 12 as a space-holder in a breast during an augmentation or reconstruction procedure using a permanent breast implant that is not included in kit 110. Kit 110 can be used in conjunction with a permanent breast implant that is procured separately from kit 110. In this example, kit 110 also does not include composition 26.

Although not shown, other components useful for facilitating the surgical procedure may also be included in kit 10 or 110, for example, peripheral components such as fat collection or delivery instruments or components for facilitating transfer of fat to the syringe 18.

### EXAMPLE

A 36-year old healthy woman desires a trimmer abdomen and larger breasts and is approved by her physician to undergo a implant and fat grafting combination surgery.

The surgeon, preoperatively with the patient's approval, identifies potential donor sites for fat graft harvest, including the lower abdomen and hips. Anesthesia is infiltrated into the donor sites for fat graft harvest about 10-15 minutes before liposuction is performed.

A breast incision is placed in a natural breast fold of the right breast to limit visibility. A subgladular pocket is made in the breast to receive the mock implant and, later in the procedure, the permanent implant.

The mock implant is removed from the hermetically sealed package and introduced and positioned in the surgical pocket, for example, using techniques known by the surgeon for implanting a permanent implant. The incision is sealed using suturing.

A 3-mm Coleman aspiration cannula is then used to harvest adipose tissue from the donor site with manually generated negative pressure. Harvested fat is transferred to 10 cc syringes and centrifuged at about 3000 rpm for about 3 minutes. Supernatant oil is removed from the lipoaspirate and fluid at the dependent portion of the syringe decanted. The fat grafts are then transferred into 1 ml and 2.5 ml syringes for transfer into breast tissue of the implanted breast in regions where divots or depressions appear, or where more volume or shaping is desired, for example, in accordance with the patient's desired aesthetic outcome. Introduction of the fat grafts is done by means of blunt tipped cannulas. The fat is injected through multiple passes and different tissue planes to improve graft take, overcorrecting defects from about 20 to about 25%. In the event that the mock implant is contacted with the tip of the blunt cannula during the fat grafting procedure, due to the reinforced nature of the mock implant, the mock implant is not pierced, torn or otherwise damaged.

The incision is then reopened and the mock implant is removed from the breast. Any injectable material that has entered the surgical pocket is removed therefrom. A permanent breast implant is then placed in the surgical pocket. Afterward or concurrently, the procedure is repeated on the left breast. The surgeon completes the augmentation surgery in accordance with conventional practice.

An aesthetic analysis is performed using preoperative and postoperative digital photographs. Follow-up visits are performed after 1 month, 3 months, 6 months and 12 months. At the follow up visits, the patient reports full satisfaction with the aesthetic outcome of the surgery.

Unless otherwise indicated or otherwise clearly contradicted by context, combinations of the above-described elements in all possible variations thereof are contemplated to be included within the scope of the invention.

What is claimed is:

1. A method for facilitating a breast augmentation or reconstruction procedure comprising:
performing an incision in a breast of a patient and forming a surgical pocket in the breast;
introducing, into the pocket, a mock breast implant;
introducing an injectable composition into breast tissue adjacent the mock breast implant in the breast;
replacing the mock implant with a permanent breast implant; and
sealing the surgical incision with the injectable composition and permanent breast implant in place in the breast.

2. The method of claim 1 wherein the step of introducing an injectable composition comprises introducing a composition comprising adipose tissue.

3. The method of claim 1 wherein the step of introducing a injectable composition comprises introducing a hyaluronic acid-based soft tissue filler composition.

4. The method of claim 1 wherein the step of introducing a injectable composition comprising introducing a blunt tipped cannula.

5. The method of claim 4 wherein the mock implant is a device that is resistant to damage by the blunt tipped cannula.

6. The method of claim 1 further comprising removing injectable composition from the surgical pocket prior to the step of replacing the mock implant with the permanent implant.

7. A kit for use in breast augmentation or reconstruction, the kit comprising:
a mock breast implant; and
written or electronic instructions to use the mock implant as a space-holder in a breast.

8. The kit of claim 7 further comprising an assembly for facilitating adipose tissue grafting in the breast.

9. The kit of claim 7 wherein the device is a syringe and blunt tipped cannula.

10. The kit of claim 7 further comprising a permanent breast implant.

11. The kit of claim 10 wherein the permanent breast implant is about the same size and shape as the mock breast implant.

12. The kit of claim 10 wherein the mock breast implant and the permanent prosthesis are constructed of different materials.

13. The kit of claim 10 wherein the permanent implant is a silicone gel filled implant.

14. The kit of claim 7 wherein the mock implant comprises a saline-fillable elastomeric envelope.

15. A kit for use in breast augmentation or reconstruction, the kit comprising:
a mock breast implant;
a permanent breast implant;
a fat grafting device; and
instructions to use the mock implant as a space-holder in a breast during a fat grafting procedure, the mock implant to be replaced with the permanent implant after the fat grafting procedure.

16. The kit of claim 15 wherein the mock implant is structured to have an appropriately matched size relative to the permanent implant.

17. The kit of claim 15 wherein the mock implant comprises a saline-fillable elastomeric envelope.

18. The kit of claim 15 wherein the mock implant comprises a saline-fillable elastomeric envelope having an appropriately matched size relative to the permanent implant.
